(19) United States
(12) Patent Application Publication
(10) Pub. No.: US 2007/0161711 A1
(43) Pub. Date: Jul. 12, 2007

(54) Process for producing pellets for pharmaceutical compositions

(75) Inventors: Christopher Speirs, Leatherhead (GB); Peter Moir, Country Waterford (IE); Richard Williams, Basel (CH); Michael Clark, Gloucestershire (GB)

Correspondence Address: BACON & THOMAS, PLLC, 625 SLATERS LANE, FOURTH FLOOR, ALEXANDRIA, VA, 22314, US

(73) Assignee: TEMREL LIMITED, DOUGLAS (GB)

(21) Appl. No.: 10/581,595

(22) PCT Filed: Dec. 15, 2004
(86) PCT No.: PCT/GB04/05263

§ 371 Date: Feb. 23, 2007

(30) Foreign Application Priority Data
Dec. 23, 2003 (GB) 0329851.0
Dec. 23, 2003 (GB) 0329854.4

(51) Int. Cl. A61K 31/12

(52) U.S. Cl. 514/678

(57) ABSTRACT

Water is used to control particle size in a process comprising mixing water with a composition comprising a rheology modifying agent and possibly sugar and cellulose to produce a paste. The paste is extruded to form particles which are then spheronised and dried. One advantage of using water to control particle size is that the number of particles having a diameter within required range, e.g. between from about 800 to about 1500 μm, may be increased.

The present invention relates to a process to produce particles, particularly for use in pharmaceutical compositions. In particular, the invention relates to the use of water to control particle size.

U.S. Pat. No. 5,834,021 (Speirs; published on 10 Nov. 1998) discloses a non-disintegratable solid enteric composition comprising 5 wt % prednisolone metasulphobenzoate (“Pred-MSB”) in an excipient matrix comprising 40 wt % microcrystalline cellulose, 35 wt % lactose and 20 wt % croscarmellose sodium. The composition is in the form of pellets having a diameter in the range of 1000 to 1400 μm. The pellets are formed by dry mixing the Pred-MSB with the cellulose, the lactose and the croscarmellose sodium. Water is added to the mixture which is then stirred for 10 minutes to form an extrudable paste. The paste is extruded from a 25 mm diameter bowl through a 1 mm diameter tube of about 5 mm length at a rate of about 100 mm/min and spheronised on an 8 in (20 cm) plate rotated at about 1000 rpm for 10 to 15 minutes to provide said pellets. The resultant pellets are dried at 50° C. for 30 min on a fluidised bed. The pellets are then coated with an Eudragit™ S100 (available from Röhm Pharma GmbH, Darmstadt, Germany) coating to provide a theoretical weight gain on coating of 11.6% and filled (15.7 mg per capsule) into size 1 hard gelatin capsules. The filled capsules are coated with an Eudragit™ L100 (also available from Röhm Pharma GmbH) coating to provide a theoretical weight gain on coating of 10.2%. The coated capsules may be used as a delayed and sustained release oral treatment of inflammatory bowel disease (“IBD”).

Similar treatments of IBD are described in UK patent application Nos. 0215656.0 and 0215657.8 (Speirs; unpublished). The contents of U.S. Pat. No. 5,834,021, GB0215656.0 and GB0215657.8 are incorporated herein by reference.

The diameter of the pellets is usually in the range between from about 500 to 2500 μm, preferably 800 to 1700 μm, more preferably 800 to 1500 μm and still more preferably 1000 to 1500 μm. However, it should be appreciated that pellets may have a diameter anywhere within the aforementioned ranges and that a capsule may have pellets having a range of diameters. One reason pellets of this size are preferred is that they may be coated satisfactorily with, for example, an enteric coating. Such enterically-coated pellets display the required release profile in the intestines. Smaller pellets tend to be less spherical and more elongated and may be below the required size to allow homogeneous filling of capsules while retaining a sufficient number to distribute through the bowel. The preferred size ranges have been justified by bioscintigraphy, the results of which showing that 200 or so pellets obtained an appropriate spread throughout the bowel.

The process disclosed in U.S. Pat. No. 5,834,021 produces a range of pellet sizes. The pellets have to be screened so that the pellets of required size can be collected. Pellets that are either too large or too small to be used effectively in the delayed and sustained release capsules would normally be discarded resulting in significant wastage. Such wastage is obviously undesirable. There is a need therefore for an improved process that produces particles having a more favourable distribution of particle sizes that is more particles within the required diameter range, resulting in a reduction in the amount of wastage.

The inventors have discovered that even small variations, e.g. ±5 wt %, in the amount of water used in the above-mentioned process causes a significant change in the size of the particles and the distribution of particle sizes. With this in mind, the inventors reasoned that particle size and, more importantly, particle size distribution is dependant on the amount of water used. The inventors realised that the amount of water could, therefore, be used to control the particle size and distribution. In this way, particles having different ranges of sizes could be produced.

According to a first aspect of the present invention, there is provided use of water to control particle size in a process for the production of particles for use in a pharmaceutical composition, said process comprising:

mixing water with a component composition comprising at least a rheology modifying agent to produce a paste;

extruding at least a portion of the paste to form extrudate;

spheronising at least a portion of the extrudate to form spheronised particles; and

drying at least a portion of the spheronised particles,

The term “paste” is intended to include wet granulate.

The particles of the present invention are typically pellets or granules. In preferred embodiments, the composition further comprises sugar and cellulose.

Without wishing to be bound by any particular theory, the amount of water affects particle size due to the state of hydration of the matrix of the particle. Once the amount of water passes a certain point, the matrix is too wet and forms large agglomerates. It would appear that a large amount of water is taken up by the rheology modifying agent. Beyond the saturation point for this process, the amount of water appears critical.

One advantage of the present invention is that more particles having a diameter within the required range, usually 800 to 1500 μm, are produced. Different pluralities of preferred pellets of this size may be treated/coated using different modalities or thicknesses of delayed release coating material in order to achieve release at specified areas of the bowel. An example of such coated pluralities of pellets is disclosed in PCT/GB03/02911, the disclosure of which is incorporated herein by reference.

Such coated pluralities of pellets allow a number of clinical objectives to be met. For example, they allow continuous delivery of a drug to treat large areas of bowel where the drug would otherwise be absorbed or metabolised if suddenly released. In addition, they allow continuous delivery of a drug over a section of the bowel to increase contact with the absorptive mucosa thereby allowing maximum absorption whereas the drug would be broken down if otherwise released in one section. Further, where a drug at high concentration would be toxic to the gut mucosa, the pellets allow the drug to be continually available at low concentration thereby allowing absorption without or with reduced toxicity.

Water is usually used in an amount of between from about 180 wt % to about 190 wt % of the component composition and is preferably used in an amount of about 185 wt % of the component composition. The inventors found the amount of water used in the process to form the matrix by absorption to be surprisingly large. This large amount of water distinguishes the present invention over all other pelletting processes of which the inventors are aware.

Usually from about 80% to about 98% of particles and, typically between from about 90% to 98% of particles, have a diameter between the range of about 800 to about 1500 microns. Even though the number of particles whose diameter is within the required range is greater than for the process disclosed in U.S. Pat. No. 5,834,021, the dry particles may be screened to obtain particles having a diameter with the range of about 800 to about 1500 μm and to remove particles whose diameter does not fall within that range.

Use of about 5 wt % less water usually reduces particle size significantly. Conversely, use of 5 wt % more water increases particle size such that 100% of particles have a diameter greater than 1500 μm which is useless if the pellets are to be enterically coated and used to release an active into the bowels.

The use of less water reduces the particle size distribution such that fewer particles have a diameter within the desired 800 to 1500 μm range and the mean particle size is reduced. The use of more water increases particle size distribution until all pellets are greater than 1500 μm. Thus, preferred embodiments of the present invention increase the number of useful particles and reduces the amount of waste.

Pellets produced according to the invention are particularly applicable to the delivery of high molecular weight compounds, for example proteins or peptides, in which the integrity of the tertiary structure is critical to the efficacy and safety of the compound. A particular advantage of these pellets is that an oral pharmaceutical composition may be prepared under gentle conditions relative to most pharmaceutical processes, whilst providing a desired release profile of the compound in the intestinal tract.

An example of a high molecular weight compound, which would benefit from formulation in a composition of the present invention is erythropoietin, a glycosylated protein hormone and haematopoietic growth factor, which is considered useful in the management of anaemia in chronic renal failure among other conditions and has been investigated in the treatment of anaemia of inflammatory bowel disease as well as other normocytic-normochromic anaemias. Erythropoietin is conventionally administered subcutaneously or intravenously, although a tabletted form of erythropoietin has been disclosed (RU-A-2152206).

Other classes of high molecular weight compound which may benefit from the present invention include interferons, TNF antagonists and specific protein and polypeptide agonists and antagonists of the immune system, hormones, such as human growth hormone and cytokines and cytokine antagonists. Other high molecular weight compounds that might be used include vaccines.

Particles produced according to the invention are also particularly useful in the delivery of anti-infective compounds such as metronidazole. Such pellets achieve high concentrations of the anti-infective compounds in the lumen of the gut and at the gut wall and allow the anti-infective agent to be disseminated through an appropriate extended area of the gut. In addition, pellets comprising an anti-inflammatory agent also achieve a high concentration of the agent in the gut wall.

Other compounds and classes of compound whose administration may benefit from the present invention include analgesics and antipyretics; antibacterial and antiprotozoal agents, such as metronidazole, albenazole, mebendazole, prazinquantel and other nitroimidazole antibiotics and antibiotics active against anaerobic bacteria; clarithromycin and other macrolide antibiotics; gentamycin, ciprofloxacin, rifabutin and other such antibiotics active against infective organisms commonly associated with or causing disorders of the intestine; antifungal agents; antiinflammatory agents such as, salicylates, for example 5-aminosalicylic acid, 4-aminosalicylic acid and derivatives, such as balsalazide, steroids, especially prednisolone metasulphobenzoate; probiotics and prebiotics which have been shown to influence the symptoms of inflammatory bowel disease and irritable bowel syndrome and recovery from antibiotic-associated diarrhea. Similarly, pharmacologically active drug substances known to influence the symptoms of irritable bowel syndrome, particularly by affecting neurotransmission in the gut at local sites such as those affecting the serotinergic system and those active at the site of opiate receptors. α-amylase and paracetamol may also be administered using the composition of the present invention.

Other compounds which may benefit from the present invention include certain compounds that have toxic effects which limit their clinical usefulness, especially by causing local toxicity in specific areas of the gastrointestinal tract. Included among such compounds are examples of antibiotics, bisphosphonates and antiinflammatory drugs. A particular example is metformin, which is intolerable to many patients due to adverse effects on the gastrointestinal tract. The present invention may be utilised to minimise the concentration of the compound at the specific sites of toxicity and so allowing an effective therapeutic dose to be administered with a reduction in adverse events.

Antibiotics effective in the treatment of inflammatory bowel disease or infective disorders of the intestine are frequently toxic when absorbed and the present invention may be applied to administer them to their sites of action in the intestine, achieving sufficient local concentrations whilst minimising systemic uptake. Of particular application to the present invention are toxic antibiotics, such as gentamycin, particularly in patients predisposed to the toxic effects of such drugs such as those with renal dysfunction. Patients with chronic disorders of the intestine, for example Crohn's disease and pouchitis, requiring continued administration of certain antibiotics, for example, metronidazole, over long periods are likely to benefit particularly from the present invention.

Other possible actives include anticancer or cytotoxic agents such as cyclophosphamide, cisplatin and other platinum drugs and vincristine and other vinca alkaloids; immunomodulators such as methotrexate, azathioprine and cyclosporin; and anti-parasitic agents such as albenazole.

Pharmacologically acceptable salts and derivatives of the active compounds may also be used.

The preferred compounds for use in the present invention are prednisolone sodium metasulphobenzoate, 5-aminosalicylic acid, metronidazole, clarithromycin, metformin, paracetamol, α-amylase and erythropoietin. In the case of prednisolone pellets, the particles may be used to treat inflammatory bowel disease, for example, in a delayed and sustained release oral medicament.

The therapeutically active compound is preferably present in a therapeutically effective amount, usually between from more than 0 wt % to about 90 wt %, preferably between from more than 0 wt % to 40 wt %, of the component composition. The final amount of the active depends on the potency of the active. Therefore, actives that have relatively higher potency, for example erythropoietin, may be present in an amount between from more than 0 wt % to about 1 wt %. In addition, actives that have relatively lower potency, for example prednisolone or metronidazole, may be present in an amount between from about 5 wt % to about 20 wt %.

One preferred composition consists essentially of prednisolone or a pharmacologically acceptable salt (e.g. predisolone sodium metasulphobenzoate) or derivative thereof, rheology modifying agent, sugar and cellulose.

A second preferred composition consists essentially of metronidazole or a pharmacologically acceptable salt or derivative thereof, rheology modifying agent, sugar and cellulose.

A third preferred composition consists essentially of erythropoietin or a pharmacologically acceptable salt or derivativre thereof, rheology modifying agent, sugar and cellulose.

The rheology modifying agent is swells upon hydration to form a gel-like matrix having visco-elastic properties. When the pellets are dried, they do not shrink significantly. Therefore, the Inventors reason that, once the water is removed, a particular structure is formed which might be responsible for the release characteristics of the pellets. The rheology modifying agent is usually a hydrophilic gelling agent such as starch or hydropropyl-methylcellulose.

The rheology modifying agent may be, e.g. crospovidone, sodium starch glycolate or croscarmellose sodium, i.e. Ac-Di-Sol™ (FMC Biopolymer, 1735 Market Street, Philadelphia, Pa. 19103, USA). Croscarmellose sodium is usually used as a super disintegrant, i.e. a compound that assists dissolution of a composition. It is, therefore, surprising and totally unexpected that a super disintegrant would form a gel-like matrix. The rheology modifying agent is present in an amount of at least 5 wt % of the component composition, preferably at least 10 wt % and more preferably in an amount of between from about 10 to about 40 wt %, e.g. 20 wt %, of the component composition.

The sugar is preferably lactose monohydrate. The sugar is preferably present in an amount of between from about 30 to about 50 wt %, e.g. 35 wt %, of the component composition.

The cellulose is preferably microcrystalline cellulose. The cellulose is preferably present in an amount of between from about 35 to about 45 wt %, e.g. 30 wt %, of the component composition.

The speed of the spheroniser is very slow in comparison to that in known pellet manufacturing processes. For the purposes of the present invention, the spheronising plate usually rotates at between from about 125 rpm to 1800 rpm, preferably 200 rpm to 1000 rpm and, if the speed of rotation used is outside this range then the spheroniser usually fails to make pellets. In addition, with knowledge of known processes, the use of a smaller spheronising plate would intuitively require a faster rotation speed. However, in the present invention, the reverse is true and a smaller plate requires a faster speed of rotation. To the inventors' knowledge, this observation is unique in pellet manufacturing.

Controlling the amount of water used allows optimisation of the size distribution of particles at maximum process yields. The particles are intended for a particular purpose, for example medical treatment of a condition, e.g. IBD.

The resultant particles may be coated with an enteric coating such as Eudragit™ S which is an anionic copolymer of methacrylic acid and methacrylic acid methyl ester in which the ratio of free carboxylic groups to ester groups is approximately 1:2 and has a mean molecular weight of 135,000. A plurality of the coated particles may be encapsulated in a capsule or compressed into a tablet. The capsule or tablet may be coated with another enteric coating such as Eudragit™ L which differs from Eudragit S in that the ratio of free carboxylic groups to ester groups is approximately 1:1. Both Eudragit™ L and Eudragit™ S are insoluble in gastric juice (about pH 6) but only Eudragit™ L is readily soluble in intestinal juice below about pH 7. In this way, release of the active component is delayed until the colon and sustained to increase the effectiveness of the active. Sustained release is believed to be achieved at least in part through the coating becoming permeable.

It is believed currently that the gel-like matrix is formed from the cellulosic components of the pellets upon rehydration. In preferred embodiments, the cellulosic components are microcrystalline cellulose and croscarmellose sodium (a cellulose derivative). On rehydration, the pellets swell and release the active component in a sustained manner over time. The pellets also become “sticky” on rehydration and stick to the gut wall. As a result, the swollen pellets stick to the target site in the gut thereby increasing the effectiveness of the active. In addition, the pH within the gut increases from the centre of the gut lumen to the wall of the gut. Where the pellets are coated with a pH dependent release coating material, the rate of release of the active increases as the pellets approach the gut wall. This feature of preferred embodiments of the invention may also increase the effectiveness of the active.

The results also indicate that the overall yield (after drying) of the particles increases as the amount of water used approaches the optimum amount.

In a second aspect of the present invention, there is provided a process for the production of particles for use in a pharmaceutical composition, said process comprising the steps of:

mixing water with a component composition comprising at least a rheology modifying agent to produce a paste;

extruding at least a portion of the paste to form extrudate;

spheronising at least a portion of the extrudate to form spheronised particles; and

drying at least a portion of the spheronised particles.

Preferably, the amount of water used is between from about 180 to about 190 wt % of the weight of the component composition and, where the spheronising step uses a rotation 70 cm plate, the plate does not rotate at about 33 rpm.

The process of the second aspect may have any or all of the preferred features of the process defined above, in any appropriate combination.

Preferred embodiments of the present invention will now be described, by way of example only and with reference to the accompanying figures. In the figures:

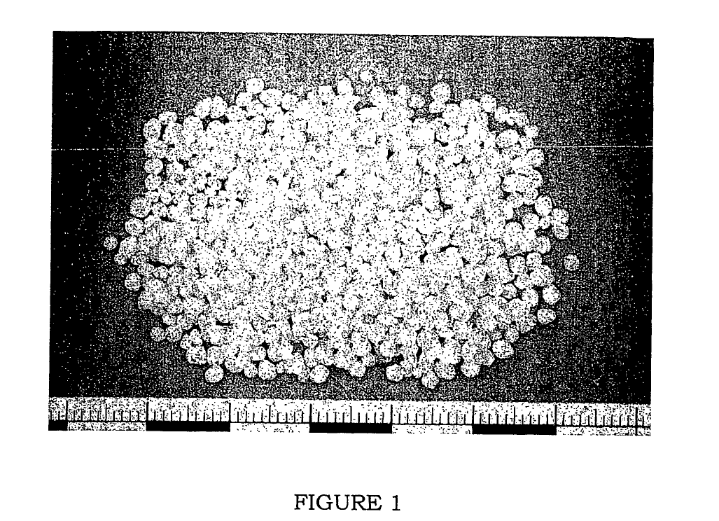

FIG. 1 is a photograph of uncoated pellets produced in Example 1;

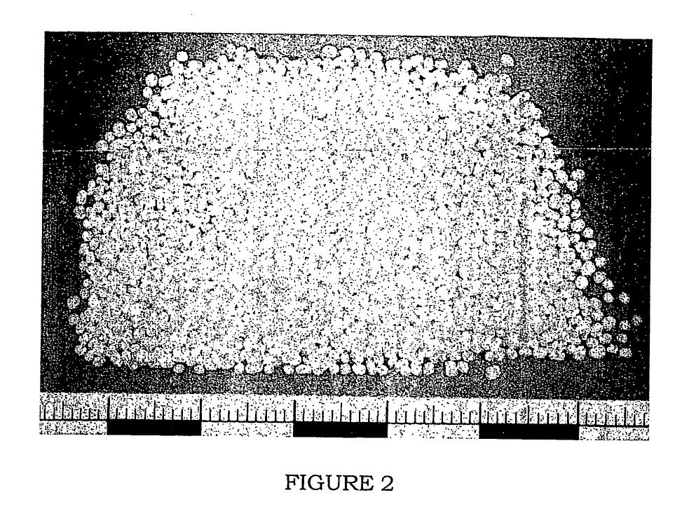

FIG. 2 is a photograph of uncoated pellets produced in Example 2; and

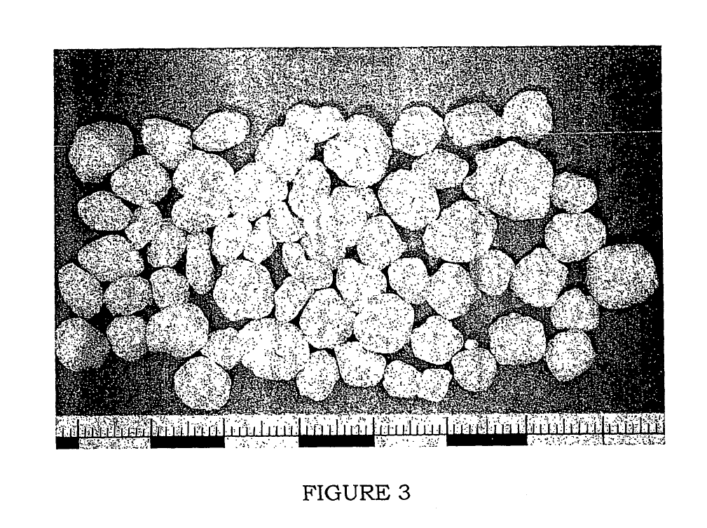

FIG. 3 is a photograph of uncoated pellets produced in Example 3.

### EXAMPLE 1

5 wt % Prednisolone Sodium Metasulphobenzoate

Prednisolone metasulphobenzoate pellets were prepared by preparing a dry mix of 5 wt % prednisolone sodium metasulphobenzoate, 40 wt % microcrystalline cellulose (Avicel™ PH 101), 35 wt % lactose monohydrate (D80 200 Mesh) and 20 wt % croscarmellose sodium (Ac-Di-Sol™). Purified water (185 wt % of the dry mix components) was added and the resulting mixture mixed for 10 minutes to form and extrudable paste which was then extruded and spheronised. The pellets were then dried in a fluid bed granulator and screened to ensure the size of the particles was in the range 800 to 1500 μm.

FIG. 1 depicts the pellets formed by Example 1. The majority of these pellets are within the required range of 800 to 1500 μm.

### EXAMPLE 2

5 wt % Prednisolone Sodium Metasulphobenzoate

Pellets were formed using the steps described in Example 1 although only 180 wt % water was used instead of 185 wt %. The yield (after drying) of the pellets was 91%.

FIG. 2 depicts the pellets formed by Example 2. The photograph clearly shows that the size of the pellets is reduced significantly when less water is used.

### EXAMPLE 3

5 wt % Prednisolone Sodium Metasulphobenzoate

Pellets were formed using the steps described in Example 1 although 190 wt % water was used instead of 185 wt %.

FIG. 3 depicts the pellets formed by Example 3. The photograph clearly shows that the size of the pellets is increased significantly when more water is used.

### EXAMPLE 4

5 wt % Prednisolone Sodium Metasulphobenzoate

Pellets were formed using the steps described in Example 1 although only 182.5 wt % water was used instead of 185 wt %. The yield (after drying) of the pellets was 96.5%.

### EXAMPLE 5

5 wt % Prednisolone Sodium Metasulphobenzoate

Pellets were formed using the steps described in Example 1 although only 177.5 wt % water was used instead of 185 wt %. The yield (after drying) of the pellets was 85%.

### EXAMPLE 6

20 wt % Metronidazole

A batch of dry mix consisting of 0.50 kg metronidazole, 1.00 kg microcrystalline cellulose (“MCC”), 0.50 kg lactose and 0.50 kg croscarmellose sodium (Ac-Di-Sol™) was prepared. The optimal amount of water for the dry mix was determined to be 5.10 kg. 90% (4.59 kg) of the optimal amount of water was added to the dry mix and a portion of the resultant mixture processed as in Example 1. After processing, a small sample of the resultant pellets was retained and the remaining pellets returned to the remaining portion of the mixture. A further 5% (0.26 kg) of the optimal amount of water was mixed with the mixture and a portion of the new mixture processed as in Example 1. This procedure was repeated a further three times so that results of pellet production runs were obtained for mixtures having 90 wt %, 95 wt %, 100 wt %, 105 wt % or 110 wt % of the optimal amount of water. The results of the five pellet production runs are indicated in Table 1.

TABLE 1

| Formulation | | Results | | | |
|---|---|---|---|---|---|
| Material | Amount (kg) | Mixture | Extrudate | Pellets | Processing |
| MCC | 1.00 | a) 90% | Looked | Smaller than | Normal. |
| | | water | normal. | normal. | |
| | | added. | | Retained | |
| | | | | sample. | |
| Ac-Di-Sol* | 0.50 | b) Added | Looked | Smaller than | Normal. |
| | | further 5% | normal. | normal. | |
| | | water. | | Visible | |
| | | | | difference. | |
| | | | | Retained | |
| | | | | sample. | |
| Metronidazole | 0.50 | c) Added | Normal. | Good, | Normal. |
| | | further 5% | | slightly | |
| | | water. | | larger than | |
| | | | | previous | |
| | | | | run. | |
| | | | | Retained | |
| | | | | sample. | |
| Lactose | 0.50 | d) Added | Longer than | Larger than | Slightly |
| | | further 5% | normal | normal | more |
| | | water. Wet | strands. | pellets. | sticky. |
| | | mix | | Retained | |
| | | binding. | | sample. | |
| | | Sticking in | | | |
| | | lumps. | | | |
| Water | 5.10 | e) Added | Very long | Very large | Sticks to |
| | (100%) | further 5% | strands, | and uneven. | equipment. |
| | | water. More | | | |
| | | lumpy, more | | | |
| | | sticky. | | | |

*Crosscarmellose Sodium

The results indicate not only that pellets comprising an active component other than prednisolone sodium metasulphobenzoate may be made and but also that the size of metronidazole pellets may be controlled by controlling the amount of water present. In this connection, the results further indicate that each increase in the amount of water, increases the average size of the pellets produced.

### EXAMPLE 7

40 wt % Metronidazole (No Lactose)

A batch of dry mix consisting of 1.00 kg metronidazole, 1.00 kg MCC and 0.50 kg croscarmellose sodium (Ac-Di-Sol™) was prepared. The dry mix of Example 7 was similar to that of Example 6 except that the lactose in Example 6 was replaced with further metronidazole. The optimal amount of water for the dry mix was again determined to be 5.10 kg. ˜84% (4.3 kg) of the optimal amount of water was added to the dry mix and a portion of the resultant mixture processed as in Example 1. After processing, a small sample of the resultant pellets was retained and the remaining pellets returned to the remaining portion of the mixture. A further ˜10% (0.5 kg) of the optimal amount of water was mixed with the mixture and a portion of the new mixture processed as in Example 1. After processing, a small sample of the resultant pellets was retained and the remaining pellets returned to the remaining portion of the mixture. A further ˜6% (0.3 kg) of the optimal amount of water (total 100%) was mixed with the mixture and a portion of the further new mixture 15 processed as in Example 1. The results of the three pellet production runs are indicated in Table 2.

TABLE 2

| Formulation | | | | | |
|---|---|---|---|---|---|
| | Amount | Results | | | |
| Material | (kg) | Mixture | Extrudate | Pellets | Processing |
| MCC | 1.00 | a) 4.3 kg | Looked | Smaller | Normal. |
| | | water | normal. | than | |
| | | added. | | normal. | |
| | | | | Retained | |
| | | | | sample. | |
| Ac-Di-Sol* | 0.50 | b) Added | Looked | Slightly | Normal. |
| | | further | normal. | smaller | |
| | | 0.5 kg | | than | |
| | | water. | | normal. | |
| | | | | Retained | |
| | | | | sample. | |
| Metronidazole | 1.00 | c) Added | Normal. | Good, | Normal. |
| Water | 5.10 | further | | normal | Dried |
| | | 0.3 kg | | size. | batch |
| | | water. | | | retained. |

*Crosscarmellose Sodium

The results indicate not only that pellets comprising an active component other than prednisolone sodium metasulphobenzoate may be made and but also that the size of the metronidazole pellets may be controlled by controlling the amount of water present. As in Example 6, the results further indicate that each increase in the amount of water, increases the average size of the pellets produced.

### EXAMPLE 8

20 wt % Paracetamol

A batch of dry mix consisting of 1.00 kg paracetamol, 2.00 kg MCC, 1.00 kg lactose and 1.00 kg croscarmellose sodium (Ac-Di-Sol™) was prepared. The optimal amount of water for the dry mix was determined to be 9.50 kg. 100% (9.5 kg) of the optimal amount of water was added to the dry mix and a portion of the resultant mixture processed as in Example 1. After processing, a small sample of the resultant pellets was retained and the remaining pellets returned to the remaining portion of the mixture. A further 5% (˜0.48 kg) of the optimal amount of water was mixed with the mixture and a portion of the new mixture processed as in Example 1. After processing, a small sample of the resultant pellets was retained and the remaining pellets returned to the remaining portion of the mixture. A further 5% (˜0.48 kg) of the optimal amount of water (total ˜10.5 kg) was mixed with the mixture and a portion of the further new mixture processed as in Example 1. The results of the three pellet production runs are indicated in Table 3.

TABLE 3

| Formulation | | | | | |
|---|---|---|---|---|---|
| | Amount | Results | | | |
| Material | (kg) | Mixture | Extrudate | Pellets | Processing |
| MCC | 2.00 | a) 100% | Looked | Normal size | Normal. |
| | | water | normal. | range. | |
| | | added. | | Retained | |
| | | | | sample. | |
| Ac-Di-Sol* | 1.00 | b) Added | Looked | Slightly | Normal. |
| | | further 5% | normal. | larger than | |
| | | water. | | normal. | |
| | | | | Retained | |
| | | | | sample. | |
| Paracetamol | 1.00 | c) Added | Normal. | larger | Normal. |
| Lactose | 1.00 | further 5% | | pellets. | |
| Water | 9.50 | water. | | Retained | |
| | (100%) | | | sample. | |

*Crosscarmellose Sodium

The results indicate not only that pellets comprising an active component other than prednisolone sodium metasulphobenzoate or metronidazole may be made and but also that the size of the paracetamol pellets may be controlled by controlling the amount of water present. As in Examples 6 and 7, the results further indicate that each increase in the amount of water, increases the average size of the pellets produced.

It will be appreciated that the invention is not restricted to the details described above with reference to the preferred embodiments but that numerous modifications and variations can be made without departing from the spirit or scope of the invention as defined by the following claims.

1. (canceled)

2. The process of claim 40 wherein the water is admixed in step (a) in an amount of between from about 180 wt % to about 190 wt % of the component composition.

3. (canceled)

4. The process of claim 40 wherein between from about 80% to about 98% of particles produced in step (d) have a diameter between the range of about 800 to about 1500 μm.

5. (canceled)

6. The process of claim 2 wherein the water is admixed in an amount of about 190 wt % of the component composition and substantially all of the particles have a diameter over 1500 μm.

7. The process of claim 40 wherein the dry particles produced in step (d) are screened to obtain particles having a diameter with the range of about 800 to about 1500 μm.

8. The process of claim 40 wherein the component composition of step (a) further comprises a therapeutically effective amount of active compound selected from the group consisting of peptides, polypeptides, proteins, interferons, TNF antagonists, protein and peptide agonists and antagonists of the immune system, hormones, cytokines and cytokine agonists and antagonists, analgesics, antipyretics, antibacterial and antiprotozoal agents, anti-infective agents, antibiotics, antiviral agents, antifungal agents, antimalarial agents, anti-inflammatory agents, steroids, probiotics and prebiotics, opiate agonists and antagonists, bisphosphonates, anticancer and cytotoxic agents, immunomodulators, antiparasitic agents and pharmacologically acceptable salts and derivatives of each of these active compounds.

9. The process of claim 40 wherein the component composition of step (a) further comprises a therapeutically effective amount of active compound selected from the group consisting of erythropoietin, human growth hormone, metronidazole, albenazole, mebendazole, prazinquantel, clarithromycin, gentamycin, ciprofloxacin, rifabutin, 5-aminosalicylic acid, 4-aminosalicylic acid, balsalazide, prednisolone metasulphobenzoate, α-amylase, paracetamol, metformin, cyclophosphamide, cisplatin, vincristine, methotrexate, azathioprine and cyclosporin and pharmacologically acceptable salts or derivatives thereof.

10. The process of claim 40 wherein the component composition of step (a) further comprises a therapeutically effective amount of prednisolone or a pharmacologically acceptable salt or derivative thereof.

11. The process of claim 40 wherein the component composition of step (a) further comprises a therapeutically effective amount of metronidazole or a pharmacologically acceptable salt or derivative thereof.

12. The process of claim 40 wherein the component composition of step (a) also comprises a therapeutically effective amount of erythropoetin or a pharmacologically acceptable salt or derivative thereof.

13. (canceled)

14. The process of claim 8 wherein the active compound is present in an amount between from more than 0 wt % to about 90 wt % of the component composition.

15-17. (canceled)

18. The process of claim 40 wherein the rheology modifying agent comprises croscarmellose sodium.

19. (canceled)

20. The process of claim 40 wherein the rheology modifying agent is present in the component composition of step (a) in an amount of at least 5 wt % of the said component composition.

21-23. (canceled)

24. The process of claim 40 wherein the component composition of step (a) further comprises a sugar.

25. The process as claimed in claim 24 wherein the sugar is lactose monohydrate.

26. The process as claimed in claim 24 wherein the sugar is present in an amount of between from about 30 to about 50 wt % of the component composition.

27. (canceled)

28. The process of claim 40 wherein the component composition of step (a) further comprises a cellulose.

29. The process of claim 28 wherein the cellulose is microcrystalline cellulose.

30. The process of claim 28 wherein the cellulose is present in an amount of between from about 35 to about 45 wt % of the component composition.

31. (canceled)

32. The process of claimed 40 wherein the component composition of step (a) consists essentially of prednisolone or a pharmacologically acceptable salt or derivative thereof, a rheology modifying agent, sugar and cellulose.

33. The process of claim 40 wherein the component composition of step (a) consists essentially of metronidazole or a pharmacologically acceptable salt or derivative thereof, a rheology modifying agent, sugar and cellulose.

34. The process of claim 40 wherein the component composition of step (a) consists essentially of erythropoetin or a pharmacologically acceptable salt or derivative thereof, a rheology modifying agent, sugar and cellulose.

35. (canceled)

36. A process for the production of particles for use in a pharmaceutical composition, said process comprising the steps of:
mixing water with a component composition comprising at least a rheology modifying agent to produce a paste;
extruding at least a portion of the paste to form extrudate;
spheronizing at least a portion of the extrudate to form spheronized particles; and
drying at least a portion of the spheronized particles, the amount of water admixed in the mixing step being between from about 180 to 190 wt % of the weight of the component composition and, where the spheronizing step uses a 70 cm plate rotating at 200 to 1000 rpm.

37-39. (canceled)

40. A process for producing particles of controlled size and size distribution for use in a pharmaceutical composition, comprising the steps of
(a) admixing water with a component composition to produce a paste, the water being admixed in an amount effective to provide in step (d) spheronized particles of controlled particle size and size distribution, the component composition comprising at least a rheology modifying agent in an amount effective to form on hydration a matrix with visco-elastic property,
(b) extruding at least a portion of the paste to form extrudate,
(c) spheronizing at least a portion of the extrudate to form spheronized particles; and
(d) drying at least a portion of the spheronized particles.

41. The process of claim 9 where the active compound is present in an amount between from more than 0 wt % to about 90 wt % percent of the component composition.
